(19) United States
(12) Patent Application Publication
(10) Pub. No.: US 2022/0183625 A1
(43) Pub. Date: Jun. 16, 2022

(54) MOUTH GUARD FOR SENSING FORCES TO THE HEAD HAVING FALSE-IMPACT DETECTION FEATURE

(71) Applicant: Force Impact Technologies, Inc., Gilbert, AZ (US)

(72) Inventors: Anthony M. Gonzales, Canoga Park, CA (US); Robert M. Merriman, Gilbert, AZ (US); Susan M. Merriman, Gilbert, AZ (US); Christopher T. Cooper, Stittsville (CA); Victoria A. Smith, Tempe, AZ (US)

(21) Appl. No.: 17/553,390

(22) Filed: Dec. 16, 2021

Related U.S. Application Data
(60) Provisional application No. 63/199,264, filed on Dec. 16, 2020.

(51) Int. Cl. A61B 5/00

(52) U.S. Cl. CPC A61B 5/682; A61B 2503/10; A61B 5/7282; A61B 5/7267

(57) ABSTRACT

A mouth guard senses an impact event and generates impact data associated with the impact event. The impact data can be analyzed to determine whether the impact event is a true impact event or a false impact event. The impact data can be analyzed by a trained model. The impact data can be labeled after independently determining whether the impact event was a true impact event or a false impact event. The labeled impact data can be used to update the model.

### RELATED APPLICATIONS

This application claims priority to and the benefit of U.S. Provisional Application No. 63/199,264, filed on Dec. 16, 2020, which is incorporated herein by reference in its entirety.

### COPYRIGHT

A portion of the disclosure of this patent document may contain material which is subject to copyright protection. The copyright owner has no objection to the facsimile reproduction by anyone of the patent disclosure, as it appears in the Patent and Trademark Office patent files or records, but otherwise reserves all copyright rights whatsoever.

### FIELD OF THE INVENTION

The present invention relates generally to wearable devices for the detection of injurious concussive forces. More particularly, this invention relates to a mouth guard with on-board electronics for sensing external impact forces, and having features for detecting and determining false impacts experienced by the user that would otherwise be determined to be an impact to the head.

### BACKGROUND OF THE INVENTION

At all levels, athletics are seen as constructive methods of exercise. Sports encourage robust competition and health. Men, women, boys, and girls participate in a variety of sports and athletic activities on a formal and informal basis. Given the variety of individuals involved, there is a large number of activities and sports played by many diverse player types. Some games involve high-speed running. And some involve more physical sports with purposeful or incidental contact between players and/or fixed objects. Contact raises the potential for harm, including head and brain injury. While American football is seen as a primary cause of sports concussions and long-term brain injury, it is less known that players in other sports also experience a high-risk for head injury and brain trauma. For instance, the incidence of concussions in girls' soccer is second only to football, and the combined incidence of concussions for boys' and girls' soccer nearly matches that of football.

Virtually any forceful impact to the head or body involves some risk level for brain trauma. Head injury may occur from collision with another player, an object, or even from a fall. Impact and rotational forces to the head are the leading causes for injury. Brain injury manifests as either neural, or most often, vascular injury within the head.

It is also widely known that the risk and severity of brain injury is related to the frequency and severity of repeated head trauma. A first blow to the head may modify the risk factors for future injury. For instance, a first incidental hit may lower the threshold for injury due to a later fall to the ground. Repeated blows and impacts have a greater impact on the risk of head trauma. Even a minor blow, below the normal threshold for injury, may cause catastrophic brain injury if it follows an earlier risk-elevating first impact. Furthermore, biometric information (i.e., sex, age, height, weight, etc.) provides an additional factor that is needed to determine the impact threshold for predicting brain injury for a particular individual.

During sports play, head injury may manifest as a temporary impairment or loss of brain function. However, more severe concussions may cause a variety of physical, cognitive, and emotional symptoms. Unfortunately, some injuries cause no immediate or obvious observable symptoms, and even minor symptoms may be overlooked, especially during the excited flow of a game. The unknown consequences of prior impacts further exacerbate the risks, by failing to diagnose an injury and take corrective action.

In recent studies, the CDC estimates that about 40% to 50% of athletes will not self-report that they may have suffered a concussive blow. While some portion of these athletes who fail to report head injuries are likely out of stubbornness, the failure to report is often attributed to the player not experiencing traditional or expected concussion symptoms. Consequently, notifying the user that he or she has received a significant impact (or impacts) is necessary for the user to report the event.

Considering the high-risk of injury in various sports and activities (such as personal fitness programs), prior art solutions have not provided a solution that is flexible and precise enough for use in the myriad of routines for the entire spectrum of athletes. For instance, given the extent of electronics and monitoring systems required for head injury assessment tools, products to be worn by players often involve a skull cap or complete helmet. A helmet, while welcomed in permissive contact sports such as football, hockey and motocross, might be out-of-place for tennis, interfere with play for a sport such as soccer, and even presents an added danger on the rugby pitch. Furthermore, prior art solutions have traded accuracy for comfort, or otherwise required a comprise sacrificing either the usefulness of information or wearing compliance with the user.

Other products include multiple part pieces that are deployed at different parts of the player's body and can be cumbersome and/or complicated to employ. Additionally, other solutions do not provide a simple, customizable, single-piece portable solution.

Clinical tests have proven that the combined measurement of linear and angular acceleration has the most accurate prediction of concussion possibilities, compared to either of the measurements independent of one another. Clinical studies suggest that sensors located in a mouth guard, as opposed to an accessory on a helmet or a chinstrap, have a higher correlation to the center of gravity of the brain. This is thought to be a result of the mouth guard's placement in relation to the rear molars, which are attached to the base of the skull. Certain anatomical landmarks on the head, such as in the inner ear, are considered by some to be effective in correlating impacts to brain injury. However, due to the size and quantity of the components required to ensure proper detection, as well as the relative locations of components, the spatial arrangement and structure of such devices have been unable to achieve a useful device that is ergonomically acceptable and a comfortable fit for the user.

One problem with mouth guards having sensors is that they do not can be subject to forces that are not indicative of an impact to the head. For example, the mouth guard can be dropped from the mouth or thrown. If it is attached to a helmet, it can swing around the helmet when the helmet is removed, causing the mouth guard to hit the helmet or adjacent structures. Or the user, may simply perform a sharp bite movement with his or her teeth, which is sensed as a high force.

The present invention resolves many of these issues by providing a mouth guard with sensors and a processor with an algorithm that helps determine detections of false impacts.

All these and other objects of the present invention will be understood through the detailed description of the invention below.

### SUMMARY OF THE INVENTION

The present invention is directed to an impact sensing device and system for communicating information about impacts and user status. A mouth guard vibrates, creates a tone, sends a local signal, and/or otherwise indicates the impact to the user. In one embodiment, a piezoelectric vibration may create a tone that is only audible to the wearer via bone conduction.

In one aspect, the present invention is a mouth guard for measuring impact forces and determining possible concussive risk and brain injury. The mouth guard device is able to detect and measure the impact force to an athlete's head during activities by use of an array of motion and accelerometer sensors. The mouth guard preferably contains a sensor array, a battery, a (wireless) power receiver with charging circuit, communications system (such as a Bluetooth low-energy transceiver), a mechanical indicator (e.g., piezo transducer), and a light indicator (e.g., an RGB LED indicator). The sensors are designed to measure force, and correlate such forces with predetermined impact thresholds, preferably for linear and angular forces. Preferably, those predetermined impact thresholds may be later modified automatically while the user is participating in the activity in response to one or more impacts to the head. The system is also capable of determining impacts caused when the device is worn in place, as opposed to false detections of forces when handled in other manners (e.g., when the user drops the mouth guard).

In another aspect, a method for detecting false impacts is disclosed. The method includes generating impact data using one or more mouth guards. Each of the one or more mouth guards corresponds to a respective user of one or more users. The method further includes determine whether an impact that is experienced by at least one of the one or more mouth guards is a true impact event or a false impact event. The determination is based at least in part on the impact data.

In some cases, the method further includes selecting a model from a plurality of models, and inputting at least a portion of the impact data into the selected model. The model is configured to analyze the impact data and determine whether the impact event is a true impact event or a false impact event.

In some cases, the method further includes selecting a first model from a plurality of models, selecting a second model from the plurality of models, inputting first impact data into the first model, and inputting second impact data into the second model. The first impact data represents one or more impacts experienced by a first user engaged in a first activity. The second impact data represents one or more impacts experienced by a second user engaged in a second activity. The first activity is different than the second activity, and the first model is different than the second model. In some cases, the first model and the second model are different types of machine learning algorithms. In other cases, the first model and the second model are the same type of machine learning algorithms that are trained differently.

In another aspect, a method for detecting false impacts includes generating impact data using one or more mouth guards. Each of the one or more mouth guards corresponds to a respective user of one or more users. The method further includes inputting at least a portion of the impact data into a model. The model is configured to analyze the impact data to determine whether an impact event experienced by at least one of the one or more mouth guards is a true impact event or a false impact event. In some cases, the method further includes independently determining whether the impact event is a true impact event or a false impact event, labeling the portion of the impact data as representing a true impact event or a false impact event to form training data, and updating them model using training data.

Additional aspects of the invention will be apparent to those of ordinary skill in the art in view of the detailed description of various embodiments, which is made with reference to the drawings, a brief description of which is provided below.

### BRIEF DESCRIPTION OF THE DRAWINGS

The present invention will be described with greater specificity and clarity with reference to the following drawings, in which:

FIG. 1 illustrates a perspective view of the back side of a flexible printed circuit board assembly of an embodiment of the present invention;

FIG. 2 illustrates a perspective view of the front side of the flexible printed circuit board assembly of FIG. 1;

FIG. 3 illustrates one embodiment of a cross-sectional view of the printed circuit board used in the flexible printed circuit board assembly of FIG. 1;

FIG. 4A illustrates a left side profile view of the flexible printed circuit board assembly along the 4A-4A line in FIG. 1;

FIG. 4B illustrates a right side profile view of the flexible printed circuit board assembly along the 4B-4B line in FIG. 1;

FIG. 5 illustrates a schematic of the components of the flexible printed circuit board assembly of FIG. 1 according to one embodiment;

FIG. 6A illustrates a user's stone model that replicates his or her maxillary region and associated dentition, which is used in the process of manufacturing the inventive mouth guard;

FIG. 6B illustrates overlaying a first layer over the user's stone model in the process of manufacturing the mouth guard;

FIG. 6C illustrates embedding the flexible printed circuit board assembly of FIGS. 1-2 in the first layer that overlays the user's stone model in the process of manufacturing the mouth guard;

FIG. 6D illustrates overlaying a second layer over the first layer and the embedded flexible printed circuit board assembly in the process of manufacturing the mouth guard;

FIG. 7 illustrates the finished mouth guard from the manufacturing process of FIGS. 6A to 6D;

FIG. 8 illustrates a graphical user interface on a mobile device that provides an indication of impacts for multiple users of the mouth guard; and

FIG. 9 illustrates a flow diagram of a method for detecting false impacts.

While the invention is susceptible to various modifications and alternative forms, specific embodiments will be shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention as defined by the appended claims.

### DETAILED DESCRIPTION OF THE DRAWINGS

The drawings will herein be described in detail with the understanding that the present disclosure is to be considered as an exemplification of the principles of the invention and is not intended to limit the broad aspect of the invention to the embodiments illustrated. For purposes of the present detailed description, the singular includes the plural and vice versa (unless specifically disclaimed); the words “and” and “or” shall be both conjunctive and disjunctive; the word “all” means “any and all”; the word “any” means “any and all”; and the word “including” means “including without limitation.”

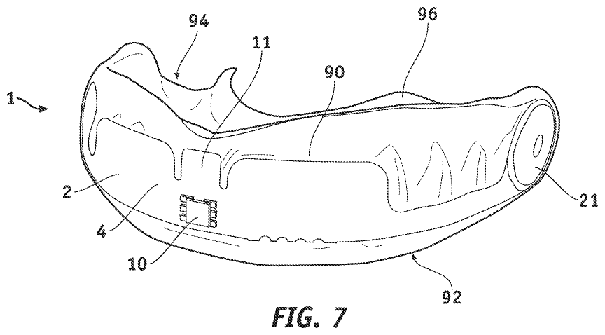

A mouth guard 1, shown best in FIG. 7, includes a flexible printed circuit board (PCB) and electronic components mounted thereon, which define a printed circuit board assembly (PCBA) 2. The PCBA 2 is designed and shaped for placement within the mouth guard 1, which is custom-fit for an athlete's mouth. The electronic components are encased within the flexible material of the mouth guard 1. The PCBA 2 is also flexible and designed with minimal structural dimensions (length, height, width, depth, etc.) for flexing and pivoting to conform to the contours of the mouth guard 1 during manufacturing and use. In one aspect, the invention herein focuses on the novel arrangement of the electronic components on the PCBA 2 to achieve efficient manufacturing, excellent operational performance, and user comfort.

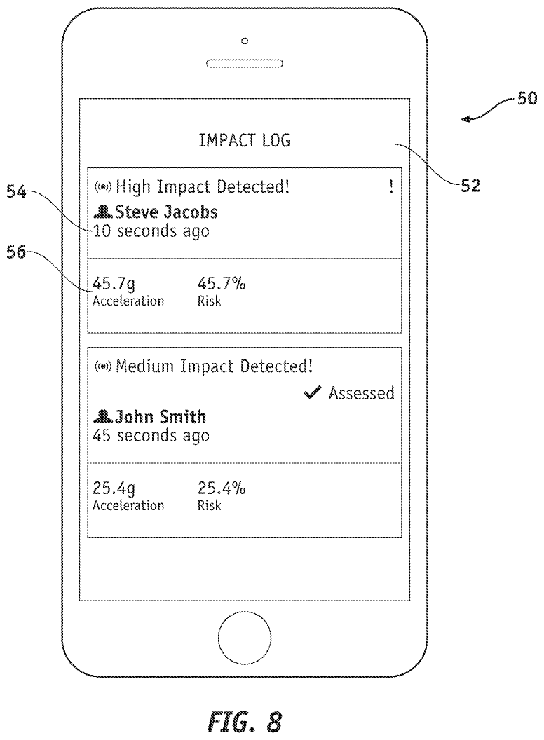

The mouth guard 1 can detect and measure impacts to an athlete's head during sports activities. An array of motion and accelerometer sensors (discussed below) detect and measure an acceleration on the user, which can then be calculated into force data. The impact data is stored on the mouth guard and may be later or contemporaneously transmitted via a transmitter (e.g., a Bluetooth low energy transceiver) to a remote smart device, such as a phone or tablet, or like device (see FIG. 8). Additional information regarding the mouth guard is disclosed in co-owned U.S. Publication No. 2017/0238850 (“Impact Sensing Wearable Device and Method”), which is hereby incorporated by reference in its entirety.

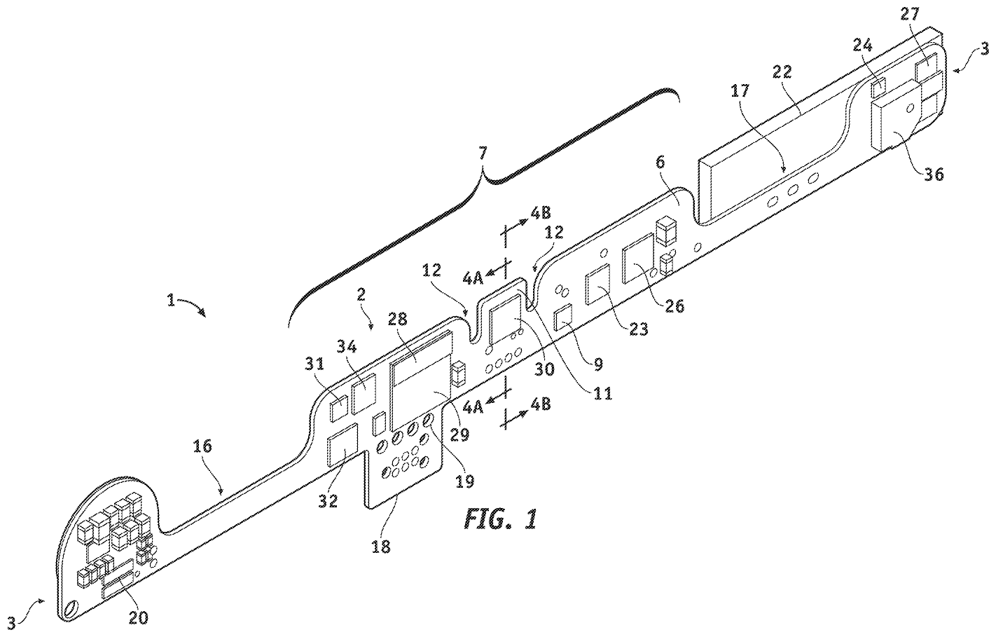

As can be seen in FIG. 1, the PCBA 2 has a length that is defined between two end portions 3 that are intended to rest along the buccal side of the molars. The PCBA 2 includes a front side 4, which will face the buccal region of the wearer after incorporation in the mouth guard 1, and a back side 6 that will face the teeth. A middle portion 7 of the PCBA 2 is positioned between the two end portions 3 and generally has a length between about 25% and 50% of the overall length of the PCBA 2. The electrical components are primarily mounted within the middle portion 7. In one preferred embodiment, the middle portion 7 is about 40% of the overall length of the PCBA 2.

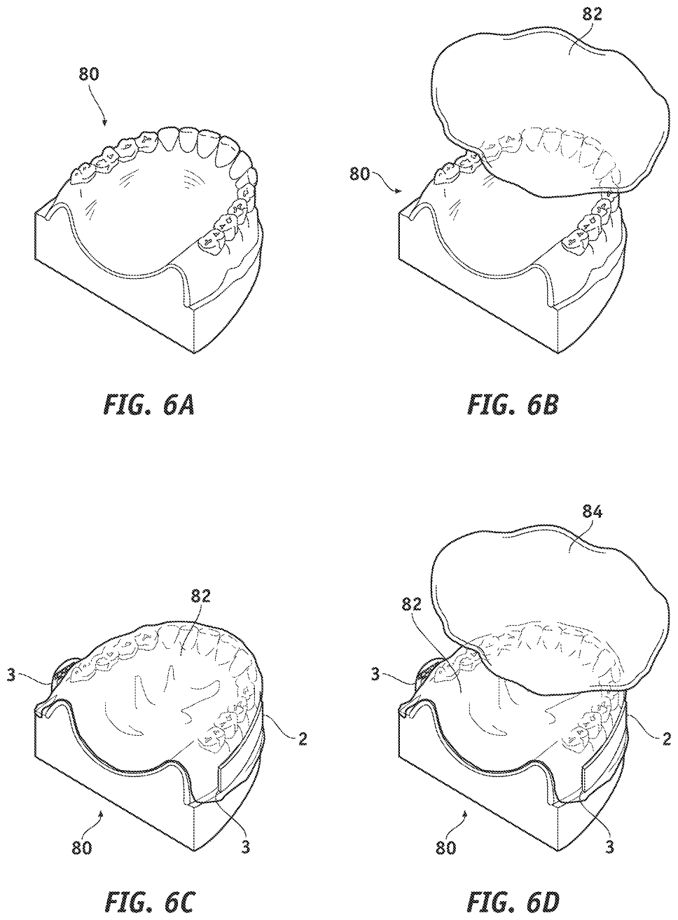

A center tab 11 is located within the middle portion 7 and is positioned in the front center of the mouth near the midline of the user's incisors. The center tab 11 is defined by a pair of cutouts 12 that provide the middle portion 7 with the ability to twist and bend as required during fabrication of the mouth guard 1, as discussed below with respect to FIG. 6. As such, the cutouts 12, which allow for dual axis rotation and twisting at pivot points adjacent thereto, allow the PCBA 2 to overcome the challenges of conforming within the custom mouth guard 1. The two cutouts 12 have a length that is at least 30% of the overall width of the middle portion 7. The middle portion 7 requires the additional bendability because it will be located along the region having the smallest radius of curvature in the dental arch (and, thus, in the mouth guard 1). The bridge portions 16, 17 of the PCBA 2 connect the end portions 3 and the middle portion 7 and are created by larger cut-outs to create a smaller dimension within the bridge portions 16, 17 for enhanced bendability of the PCBA 2.

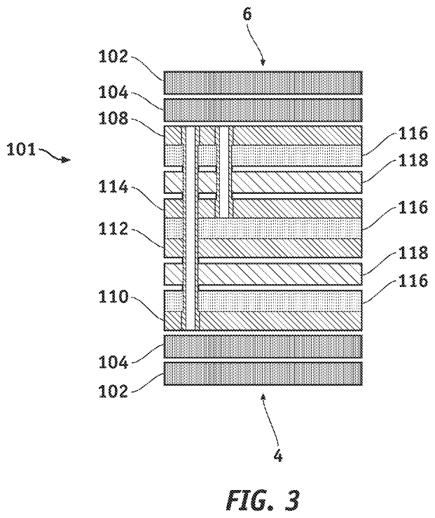

The components are preferably soldered onto PCBA 2 and an underfill (preferably a non-viscous epoxy) is used to fill in spaces within the PCBA 2 to provide some level of rigidity to the PCBA 2. The electrical components on the PCBA 2 are preferably attached with minimal solder. The largest dimension of each of the electronic components is vertically oriented when possible to accommodate bending of the PCBA 2, meaning the long edge is arranged from top-to-bottom while the short edge runs laterally along the length of the PCBA 2. Some components cannot be vertically oriented, such as a wireless charging receiver 20 and a battery 22, and, thus, are preferably placed near the end portions 3 of the PCBA 2, which will be placed along an anatomic region having a larger radius of curvature (i.e., straighter) than the middle portion 7. The PCBA 2 is preferably made of a multilayered board design to act as primary carrier of all electronic components in the mouth guard 1, as discussed below with reference to FIG. 3.

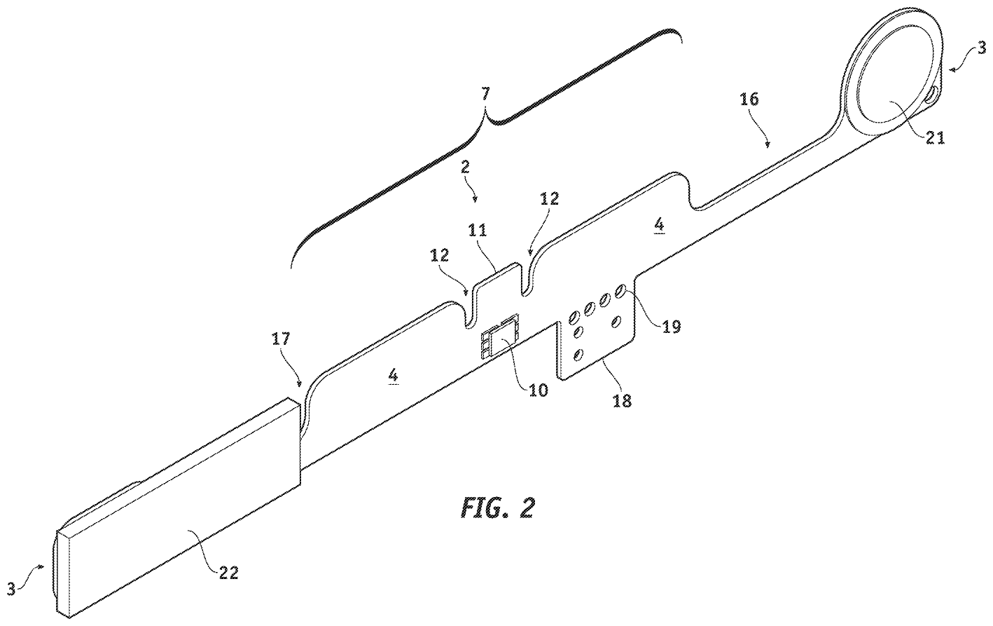

The wireless charging receiver 20 is set on the front side 4 of the PCBA 2. As shown in FIG. 2, the wireless charging coil 21 is set opposite to a charging receiver 20. The charging receiver 20 includes power circuit with inductors and capacitors, and further includes an integrated circuit controller located in proximity to and in electrical communication (e.g., wired) with the wireless charging receiver 20. The wireless charging receiver 20 and the wireless charging coil 2 are preferably set along on of the molar ends 3 and configured to best receive the energy from the external charging station (not shown). As such, the charging coil 21 may be manipulated during the manufacturing process (FIG. 6) to ensure proper positioning so as to minimize skewing relative to the peripheral side surface of the mouth guard 1 that faces the buccal surface.

Regarding the force-sensing components, the PCBA 2 includes a high-G (high-gravity) accelerometer 30, a magnetometer 31 (which includes a digital compass), and a combination low-G (low gravity) accelerometer/gyroscope 32. The magnetometer 31 and the combination low-G (low gravity) accelerometer/gyroscope 32 form an inertial measurement unit (IMU) in that they provide data that is used for sensing the orientation of the mouth guard 1. The data provided by the IMU is utilized in a sensor-fusion algorithm, which is computed in the processor to implement a sensing feature that detects the orientation of the PCBA 2 and the mouth guard 1 in three-dimensional space. The combination low-G (low gravity) accelerometer/gyroscope 32 (hereinafter “gyroscope 32”) can also be provided as two separate components, such that they are not packaged together. Alternatively, the magnetometer 31 can be packaged with the low-G (low gravity) accelerometer/gyroscope 32. The gyroscope 32 also provides data regarding angular velocity, which can be used to determine the angular acceleration (and rotational force) associated with the impact. Though these sensors 30, 31, 32 are measuring velocity and acceleration, they are herein considered to be linear and rotational force sensors because their sensed data correlates to the corresponding force and permits the processor to calculate it, as necessary.

The approximate location of the impact is computed using the IMU orientation result at the time of the impact and a calculated 3D linear acceleration impact vector. The 3D linear acceleration impact vector (relative to the mouth guard 1) is calculated using the data collected by the high-G accelerometer 30 at the moment of impact. In summary, the data received by the high-G accelerometer 30, the magnetometer 31, and the low-G accelerometer/gyroscope 32 can be used to provide information regarding the amount of linear force associated the impact, the amount of rotational force associated the impact, the spatial orientation of the impact, and movement data of the user.

Data from the high-G accelerometer 30, the magnetometer 31, and the gyroscope 32 is received and processed in a processor 28, which utilizes a memory 34 (preferably a flash memory) to correlate, relate, and otherwise store information for processing and communicating impact data. The memory 34, which may store impact data permanently or be erasable, may be located anywhere on the PCBA 2, but is preferably near the processor 28 to minimize latency. The processor 28 is further coupled with low energy Bluetooth transceiver 29. The Bluetooth transceiver 29 may include a radio and an embedded processor. The processor 28 collects data, stores the data in memory 34, and transmits the data via Bluetooth transmission, as necessary. It should be understood that while a single memory 34 is illustrated on the PCBA 2, it may have multiple memory devices 34. For example, the processor 28 may include its own memory device.

A serial wire debug (SWD) port 18 on the PCBA 2 allows wired access for initial programming of the processor(s) 28. In one embodiment, the initial programming data, such as impact thresholds based on the biometric data of the user, are stored in a memory device associated with the processor 28, while the impact data received by the sensing components is stored in the memory 34 showing in FIGS. 1-2. The user typically provides his or her biometric data when ordering the custom mouth guard 1. The biometric data of the user may include, but is not limited to: age, sex, weight, height, skull circumference, head shape (e.g., from photos of the sides, back, and/or front of the head), head mass (e.g., from a scan of the head), and/or prior concussion data. The biometric data may further include dimensional data taken from the maxilla of the user, such as from a stone model as discussed below with respect to FIG. 6. For example, the combination of the skull circumference along the forehead and the dimensional data of the maxilla provides data that correlates to the mass of the head. Alternatively, a standard mass of the head may be estimated based on the age, sex, weight, and/or height of the user using commonly known biometric information. Assuming that standard mass is known, it can then be adjusted based on measurements of the specific measurements of the user's skull and/or maxilla data. In other words, a user having a skull circumference that is 10% larger than the standard skull circumference for someone of his same age, weight, and height may have the standard mass increased by 5% due to the larger-than-standard skull circumference. Examples of linear and rotational force risk factors and threshold data for different individuals are disclosed in co-owned U.S. Publication No. 2017/0238850 (“Impact Sensing Wearable Device and Method”), which is hereby incorporated by reference in its entirety.

The biometric data of the user is used to set initial thresholds for impact forces (e.g., rotational forces and/or linear forces). In addition to establishing a single maximum threshold over which the risk of a concussion is high, the impact thresholds may include a series-based threshold that takes into account a series of impact events over a certain period of time. The series-based threshold would indicate the risk of concussion due to a series of smaller impact forces (relative to the single maximum threshold) encountered over a period of time. For example, the series-based threshold can be based on a weighted average of the hits, wherein the threshold (as measured by the weighted average) is reduced based on the number of hits. The rate of change in the reduced threshold force may be linear or exponential. These series-based thresholds are a form of dynamic thresholds, in that they change during a session (e.g., a game) of the user's activity, or over multiple sessions of activities. It should be understood that the user's prior concussion history (both short term, such as the impacts occurring over a 24-hour period, or long term, such as a prior concussion within the last two months) is also biometric data of the user that can be used to establish the thresholds. These different initial thresholds values and dynamic threshold values for the user may be stored in various look-up tables in the memory of the PCBA 2 within the mouth guard 1. And, as discussed below in FIG. 8, they may be modified over the course of operation of the mouth guard 1 by the smart device 50.

The user can also indicate different activities for which he or she intends to use the mouth guard 1. For example, a user may indicate that she intends to use the mouth guard for boxing and soccer. Each of these activities may have different impact threshold data that will be stored on the memory device of the PCBA 2 due to the different types of impact forces and frequency of impact forces that are anticipated. Some of the differences may be due to the sensitivity of the impact forces to be detected by the sensors on the PCBA 2. As one example, boxing may have hits that are longer in duration due to the deformation of the boxing gloves, whereas an undesirable head-to-head impact in a soccer match can be of very short duration. Additionally, boxing regulations may restrict certain hits, such as a hit to the back of the head. Because the mouth guard 1 can sense the directionality of hits, a hit to the back of the head in a boxing match, regardless of force, may cause the LED 10 to activate to inform the boxers (and referee) that a restricted hit occurred. Accordingly, the inventive mouth guard 1 may include impact threshold data (e.g., different look-up tables) for multiple activities of the user. The user would use a smart device (e.g., mobile phone 50 in FIG. 8) to communicate the specific activity chosen for that particular day (or session) to the mouth guard 1 via the Bluetooth connection. The processor 28 then pulls the corresponding impact threshold data stored within the memory device for the user's chosen activity and uses that impact threshold data for comparisons during the activity. It should be noted, as explained above, that two different users participating in the same two activities will still have different impact threshold data for each activity (e.g., boxing look-up table #1, soccer look-up table #1, boxing look-up table #2, soccer look-up table #2,) because of their different biometric information. Accordingly, the user's indication of his or her activity or activities (not just biometric information) may also dictate the types of impact force threshold data that are stored in the memory of the PCBA 2.

The various impact thresholds for the user and potentially other data useful for determining concussion risk can be uploaded to one of the memory devices via the SWD debug port 18. Of course, the SWD debug port 18 can be used to upload other data and software into the memory 34. Once the necessary information is loaded on the PCBA 2 and final programming is complete, the SWD debug port 18 can be removed via perforations 19 before it is encased within the flexible material of the mouth guard 1.

Because the high-G accelerometer 30 detects directional impact data, it is preferably located in a predictable reference point and, therefore, is mounted within the center tab 11 such that it is adjacent to the midline of the user's top incisors. Because its physical structure is molded into the mouth guard 1 as discussed below in FIG. 6, the high-G accelerometer 30 maintains this same position along the centerline near the biting or incisive edge line of a maxillary central incisors. In some embodiments with a user having an abnormal, or untraditional, mouth structure regarding tooth placement, fillers may be used in the mouth guard 1 to fill in gaps between teeth (for instance additional material to compensate for missing teeth, misplaced teeth, etc.). Preferably, the electrical components and, in particular, the sensors (e.g., high-G accelerometer 30, magnetometer 31, and gyroscope 32) maintain direct contact with the structure of the mouth guard 1, which, in turn, is molded to maintain direct contact with the skeletal structure and teeth of the head. As such, the sensors maintain indirect (but rigid) contact with the skeletal structure of the head. The sensors can also detect the movement and forces associated with the mouth guard 1 being dropped from the player's mouth or hand so as to discount such impact forces.

An LED driver 9 controls the actuation of an LED 10 on the front side 4 of the PCBA 2. The LED 10 is used to indicate to others (e.g., other players, a coach, a referee) that the user has experienced a certain concussion-risk event (or events when considering a series of impact forces over a period of time). The LED 10 can also indicate operation (i.e., on/off), battery-charge life, malfunction, etc. The LED driver 9 is located on front side 4 of PCBA 2 so that it can be viewed between the user's lips. In a preferred embodiment, the LED driver 9 controls both the light intensity and the color of the LED 10 via the current driven into LED 10. By supplying a fixed current, the LED driver 9 can modify that current to get the appropriate pattern of light(s) displayed on the LED 10 to indicate certain information to others located around the user (and to the user when he or she removes the mouth guard 1 from his or her mouth).

The battery 22 typically supplies between 3 volts and 4.2 volts. To maintain adequate performance levels of components, a main voltage converter 26 is used to provide a constant voltage to the components. A battery charger 23, which is coupled to the wireless charging receiver 20, preferably includes an integrated circuit to monitor and provide a specific charging profile for the battery 22. The wireless coil 21 receives alternating current, and converts it to direct current for the battery 22. The wireless coil 21 preferably receives alternating current at approximately one million hertz, or as otherwise known in the art.

The PCBA 2 includes a battery fuel gauge 24 near one of the end portions 3. The battery fuel gauge 24 utilizes a Coulomb-countering feature and a comparative table to calibrate the charge remaining on the battery 22. The battery 22 typically operates in multiple modes, such as in a normal operational mode, a charging mode, and a standby mode. The battery 22 is preferably a Lithium polymer battery with a low-profile and an ability to slightly bend during the manufacturing process (discussed in FIG. 6) to foster an ergonomic fit into the mouth guard 1. In one preferred embodiment, a gel electrolyte lithium battery is preferred, which most preferably uses a non-toxic electrolyte. While lithium polymer batteries are preferred, as battery technology improves and/or changes, other types of batteries having a thin profile and a light weight (and preferably the non-toxic nature of the electrolyte) will drive selection of battery. A battery protection component 27 may be used to prevent shorting of the battery via overcharging and/or undercharging. The battery protection 27 also prevents the battery 22 from running below a minimum charge. Because of the size of the battery 22 (length and height dimensions), the battery 22 is located toward one of the end portions 3 of the PCBA 2 in which there is more space between the buccal surface and the bone/teeth. Advantageously, the mouth guard 1 has a larger radius of curvature in this region as well, which results in less bending of the battery 22.

The PCBA 2 includes a wearer notification component 36, preferably adjacent to the end 3 of the PCBA 2. The notification component 36 uses electrical energy to generate mechanical energy to provide a haptic feedback, a vibratory feedback (e.g., a buzzer), or an auditory feedback, which may be both heard by the ear and felt within the mouth. The notification component 26 may include magnetics and/or piezoelectric elements. Because of the generation of mechanical and/or audible energy, the notification component 36 may be one of the high-energy consumption components on the PCBA 2 (the LED 10 is often the highest energy consuming component, depending on the size and functionality). Furthermore, the notification component 36 typically has the tallest profile rising from the PCBA 2 and uses a fair amount of three-dimensional space extending off of the surface of the PCBA 2. To create the most space for the notification component 36, the notification component 36 is located toward the end portion 3 of the PCBA 2 in which there is more space between the buccal surface and the bone/teeth. The notification component 36 is preferably positioned on the back side 6 of the PCBA 2 directly adjacent to the bone just below the maxillary sinus cavity to vibrate the bone and conduct vibrations along the maxilla toward the ear, which may provide a tonal sensation and/or an audible sensation (depending on the vibration frequency).

In other embodiments, the notification component 36 may use an air vibration conductor via a magnet and plate using compressed air. Alternatively, the notification component 36 may create haptic feedback through motors using offsetting weights. Alternatively, the notification component 36 may include a linear resonant actuator (LRA) using a small metal block, or pin.

The notification component 36 is particularly useful to notify the user that he or she has received a significant impact (or series of impacts) that he or she may not have recognized. For example, after a single, high-force impact creates a substantial risk of concussion, the processor 28 receives the data from the rotational and/or linear force sensing units and determines whether the predetermined threshold has been exceeded. If so, the processor 28 then communicates with the notification component 36 to begin activation that results in the haptic feedback, a vibratory feedback (e.g., a buzzer), or auditory feedback. In some embodiments, the processor 28 may delay the activation of the feedback by a set period of time (e.g., 10 or 20 seconds) such that the user has some time to regain full awareness after a big hit, so as to understand what the feedback is intended to mean. The notification component 36 can also provide different types of feedback (in duration, magnitude, or frequency) to inform the user of different events. For example, a series of lesser hits within a period of time that causes the series-based threshold to be exceeded may have a different feedback (e.g., smaller magnitude and a lower frequency) than a single, high-force impact of the component (e.g., high magnitude and a high frequency, or constant feedback). The notification component 36 can also communicate other events, such as a low-battery mode or to remind the user that system is operational, which may be accomplished in a single subtle feedback at a very low frequency (e.g., every 60 seconds).

As seen in FIGS. 1 and 2, the vast majority of the components electronics are within the middle portion 7 of the PCBA 2, but most are to the side of center tab 11 except for the LED 10 and the high-G accelerometer 30. Further, the vast majority of the sensitive electrical components are on the back side 6 of the PCBA to allow them to be impressed into the flexible material of the mouth guard 1 in a direction that faces the teeth to give them added protection from impacts to the face. As noted above, the LED 10 is on the front side 4 so as to permit the viewing of the LED 10 by other people around the user. The battery 22 is likewise on the front side 4 of the PCBA 2 for spatial reasons. The charging coil 21 is on the front side 4 to provide better access for charging the battery 22. FIGS. 1-2 represents one embodiment of the present invention and the components can be rearranged on the PCBA 2.

The width of the of the PCBA 2 (top-to-bottom) varies along the length and is between 2 mm and 15 mm, with the largest width being in the end region 3 in the illustrated embodiment. The middle portion 7 has a height in the range of 6 mm to 15 mm, and is preferably about 12 mm. Each of the end portions 3 having a width in the range of 8 mm to 15 mm. The bridge portions 16, 17 have a width that is smaller than 40% (and preferably smaller than 30%) of the widths of the end portions 3. The bridge portions 16, 17 have a width that is smaller than 50% (and preferably smaller than 40%) of the width of the middle portion 7.

FIG. 3 is a general schematic in cross-section of one preferred embodiment for a flexible printed circuit board (PCB) 101 that would receive the electrical components and form the PCBA 2. All layers are shown in FIG. 3, though not all of them are present across the entire PCB 101 as understood by those skilled in the art. Unlike prior art rigid PCBs that include foil stacked on glass or other rigid materials, the flexible PCB 101 is preferred as its shape may be modified and bent into an ergonomic position, as discussed more in FIG. 6. The flexible PCB 101 has multiple conductive layers, such as four metallic traces, that transit the signals between the electrical components. An upper and lower overlay surfaces 102 protect those internal layers, but would not present in areas when component soldering is required. Two solder-resist layers 104 are located on the metallic (e.g. copper) trace 108 of the back side 6 (primary component side) and the metallic trace 110 on the front side 4, at least in regions of the PCB 101 where component soldering is needed. The electrical working layers further include a ground layer 112 and a power layer 114 (or signal and power layer 114). Each of the four metal layers 108, 110, 112, and 114 is about 0.02 mm in thickness (e.g., 0.018 mm or 0.022 mm).

The ground layer 112 and the power layer 114 is separated by a dielectric layer 116, such as polyimide. The back side copper trace 108 is separated from the power layer 114 by a dielectric layer 116 and an adhesive layer 118. The front side copper trace 110 is also separated from the ground layer 112 by a dielectric layer 116 and an adhesive layer 118. The dielectric layers 116 and adhesive layers 118 are each about 0.025 mm in thickness. The overall thickness of the PCB 101 is between about 0.2 mm and about 0.3 mm. The PCB 101 may include exterior side tape on both the front side 4 and back side 6 in some regions, which is useful in mechanically attaching some of the larger components (e.g., the battery 22 and the charging coil 21) to the PCB 101.

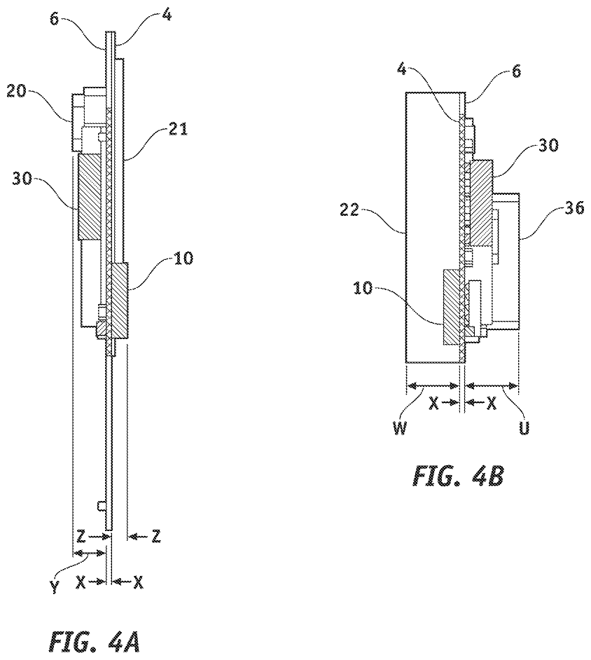

FIGS. 4A and 4B illustrate the left side and right side cross-sections of the PCBA 2 along, respectively, lines 4A-4A and lines 4B-4B of FIG. 1, both of which cut through the high-G accelerometer 30 on the back side 6 and the LED 10 on the front side 4. The dimension X-X refers to the overall thickness of the printed circuit board, which is about 0.2 mm in the illustrated embodiment. The dimension Z-Z in FIG. 4A references the height of the LED 10, which is about 0.6 mm. The battery 22 shown in FIG. 4B has a height dimension W of about 2.0 mm. The notification component 36 shown in FIG. 4B also has a height dimension U of about 2.0 mm. The charging coil 21 shown in FIG. 4A has a height dimension of about 0.4 mm, which is less than the height dimension of the LED 10. One of the parts of the coil charging receiver 20 has a height dimension Y-Y in FIG. 4 of about 1.2 mm. According, while the overall PCBA 2 has a length of about 110 mm, the maximum overall height dimension of the PCBA 2 (including components on the top side 4 and read side 6) is less than 5 mm in all regions, such that the ratio of length to overall height is greater than 20.

The end portion 3 with the battery 22 and notification component 36 may have a maximum overall height dimension of about 4.2 mm, and is preferably less than 4 mm. In the middle portion 7, the maximum overall height dimension at any point along the length is less than 2 mm (e.g., 1.8 mm), and is preferably less than 1.5 mm, which is dictated by the nearly overlaying the high-G accelerometer 30 on the back side 6 and the LED 10 on the front side 4. In the end portion 2 with the coil 21, the maximum overall height dimension is less than 2 mm (e.g., 1.9 mm), and is preferably less than 1.5 mm. The bridge portions 16, 17 of the PCBA 2 that connect the end portions 3 and the middle portion 7 preferably include no components and have a maximum dimension between about 0.2 mm to 0.3 mm, and preferably about 0.2 mm (i.e., the thickness of the printed circuit board in FIG. 3) to provide additional flexibility (both bendability and rotational twistability) to the overall PCBA 2, which is helpful when manufacturing mouth guard 1 as shown in FIG. 6. FIGS. 4A and 4B illustrate only one exemplary preferred embodiment of a low-profile PCBA 2 for use in the mouth guard 1, as other component arrangements and configurations can be used.

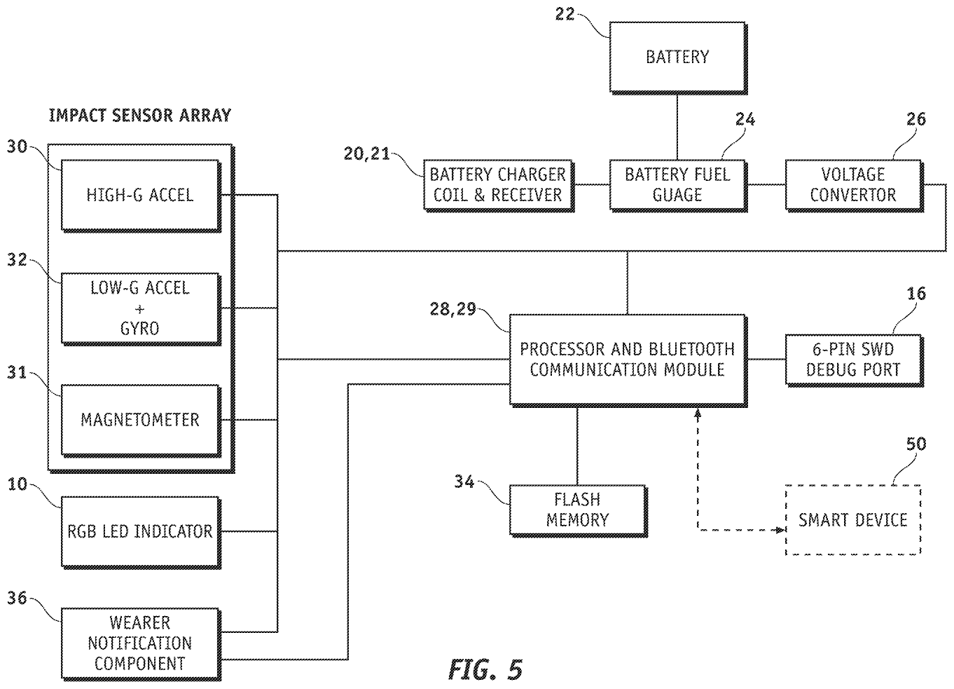

FIG. 5 is one embodiment of a general schematic illustrating the connectivity of the components mounted on the PCBA 2. The battery 22 provides power to all of the various components. The processor module includes the main processor 28 in communication with the components, receiving data (e.g., from the sensors) and sending communications instructions (e.g., to the LED 10 and notification component 36). The processor module may also contain the Bluetooth transceiver 29 that allows for communication with one or more external devices, such as the smart device 50 associated with of the user, a parent, a coach, a referee, etc. The smart device 50 would include the software (e.g., in the form of an app) to receive and transmit information with the Bluetooth transceiver 29 within the mouth guard 1 while in use or before/after use. The smart device 50 also provides remote storage, computation, and display of risk factors associated with one or more mouth guard devices. The smart device 50 may be smart phone, a tablet, or a computer. One exemplary smart device 50 is shown in FIG. 8. Other system configurations for the connectivity of the components of the PCBA 2 are possible as well.

FIGS. 6A-6D illustrate a series of stages in developing the mouth guard 1. A model 80 of the user's mouth, specifically of the maxillary region teeth, is made. The model 80 may be created by a dental professional, by use of an intra-oral scanner, or by use of a simple home impression tray. As showing in FIGS. 6A-6D, the model 80 is a stone model that is formed within an impression that was impressed over the user's teeth and gingiva. The stone is poured into the impression to create the stone model 80, resulting in a replica of the user's upper teeth and bone structure.

In one preferred embodiment shown in FIGS. 6A-6D, the method for forming the wearable mouth guard 1 is performed as follows. As shown in FIG. 6A, the model 80 is placed upside down and inserted into a thermoforming machine (e.g., a pressure thermoforming machine) that is designed to produce custom mouth guards, such as a Drufomat® manufactured and sold by Dentsply Sirona. As shown in FIG. 6B, a first base layer 82 of about 3 mm of EVA thermoplastic (other flexible materials are possible as well) is placed over the model 80. Within the thermoforming machine, the first layer 82 is heated and thermoformed over the model 80 so that it will substantially conform to the anatomical structures (e.g., bone, gingiva, teeth, etc.) of the maxillary region of the oral cavity of user, as dictated by the model 80. Due to the heat and pressure, the initial thickness of the first layer 82 decreases by about 30% to 40% when overlaid onto the model 80. For example, when the first layer 82 of 3 mm is used, the final thickness of the first layer 82 is approximately 1.8 mm to 2.1 mm. The first layer 82 is then ready to receive the PCBA 2. It should be noted that trimming and polishing may be performed on the first layer 82, as necessary.

The PCBA 2 is tested and programmed prior to being molded into the base layer 82. Once the PCBA 2 passes all tests and the latest firmware has been programmed, the region with the SWD debug port 18 (FIGS. 1-2) is trimmed off using scissors along the perforations 19. By use of the debug port 18, the memory 34 is programmed to include, for example, the specific predetermined threshold data points based on the biometric information received from that particular user.

Once the programmed PCBA 2 is ready, a heat gun is employed to sweep across and soften the first layer 82. In one embodiment, a 700 W heat gun with about 120 L/m of air flow is set to 350° C. and is used for about 30 seconds to heat the material. With the first layer 82 now softened, the PCBA 2 is placed with the back side 6 (and the majority of the electrical components) facing inward towards the softened first layer 82. The softening is not harsh enough to affect the conformance of the first layer 82 to the underlying model 80. For the first connection point, the high-G accelerometer 30 on the central tab 11 (not shown in FIG. 6) is positioned directly adjacent to the midline of the two central incisors. The central tab 11 and physical structures of the high-G accelerometer 30 and adjacent components, such as the LED driver 9 and the processor 28, on the rear side 6, are forced into and depressed within the first layer 82. For vertical alignment, the edge of the middle portion 7 closest to the LED 10 should be aligned with the cutting edges of the replica incisors within the model 80. Next, the remainder of the components on the rear side 6 of the middle portion 7 are impressed into the softened first layer 82.

After the components of the middle portion 7 are impressed within the first layer 82, the remaining portions of the left side and right side of the PCBA 2 are depressed into the first layer 82, including all components (e.g., wireless charging components 20 and the notification component 36) near the two end portions 3. Typically, the heat gun may be needed again to soften the first layer 82 along its sides to accommodate those materials. The bridge portions 16, 17 of the PCBA 2 provide the bending and twisting needed to accommodate the wide variety of anatomic variations in the maxilla region of the general population. As shown in FIG. 6C, the PCBA 2 is fixed into the first layer 82 by impressing the geometric arrangement of the electrical components on the back side 6 of the PCBA 2 into the softened first layer 82. The LED 10, the battery 21, and the charging coil 21 are mounted to the front side 4 of the PCBA 2 and, thus, project outwardly away from the underlying model 80.

Because of the unique shape of the model 80 due to each user's unique anatomy, the PCBA 2 will fit a bit differently on the first layer 82 for each user. Accordingly, a deformable filler material, such as Fillin™ from Dreve Dentamid GmbH, is provided to fill in the small gaps between the back side 6 of the PCBA 2 and the first layer 82, and also between the first layer 82 and some of the larger inwardly-facing (tooth-facing) components, such as the wireless charging components 20 and the notification component 36. This deformable material is similar to a wax such that it can be rolled into thin pieces and shaped as necessary. Additionally, the deformable filler material is used to smooth any rough surfaces (like the corners of the battery 22 or the coil 21) to prevent unnecessary pressure points for the next layer. This helps to eliminate gaps between the PCBA 2 (and the fronts-side electrical components) and the base layer 82, which may permit air to become trapped when the second layer 84 (e.g. EVA) is applied. In other words, the deformable filler material is used to fill in gaps and smoothen sharp corners for a more reliable placement of the second layer 84. The deformable filler material can also be type of hot glue, such as EVA.

As shown in FIG. 6D, the second layer 84 is placed over the combination of the first layer 82 and the PCBA 2 (and any Fillin™ material) within the thermoforming machine, to allow pressure molding of the second layer 84 over the PCBA 2. The heated second layer 84 is formed to fit over the LED 10, the battery 22, and the charging coil 21 on the front side 4 of the PCBA 2. The second layer 84 can be anywhere from 1 mm to 4 mm and, like the first layer 82, becomes thinner (and denser) upon application of heat and pressure. Assuming a 2 mm final thickness of the first layer 82, a second layer 84 of 1 mm creates about 3-4 mm thickness across most of the mouth guard 1, which may be more appropriate for sports like biking, basketball, etc. Assuming a 2 mm final thickness of the first layer 82, a second layer 84 of 4 mm creates about a 4-5 mm thickness across most of the mouth guard 1, which may be more appropriate for direct impact sports, such as boxing or football. The thickness of the second layer 84 depends in part on user preference, and the requirement for safe functionality. Trimming and polishing can be performed after the second layer 84 has been added to the first layer 82 and PCBA 2.

The total thickness, of course, varies a bit over the regions of the mouth guard 1 due to the electrical components on the PCBA 2. For example, in the front portion 92 (FIG. 7) of the mouth guard 1 having an arch-shaped surface 94 (FIG. 7) exposed to the buccal surface, the end portion 3 of the PCBA 2 having the battery 22 and notification component 36 will have the greatest localized thickness of the overall mouth guard 1, with a thickness less than 7 mm, and preferably less than 6 mm. In the front portion 92 of the mouth guard 1 exposed to the buccal surface and near the middle portion 7 of the PCBA 2 (with the majority of the components), the maximum localized thickness of the mouth guard 1 will be less than 5 mm, preferably less than 4 mm. In the front portion 92 of the mouth guard 1 exposed to the buccal surface near the other end portion 3 of the PCBA 2 having the charging coil 21 and wireless charging components 20, the maximum localized thickness of the mouth guard 1 will be less than 5 mm, preferably less than 4 mm. Between these three regions, the thickness in the bridge portions 16, 17 be less than 4 mm, or less than 3 mm.

In one example illustrating the thickness of the front portion 92 of the mouth guard 1 that faces the buccal region of the user's mouth, with the first layer 82 being 3 mm in thickness and the second layer 84 being 2 mm in thickness, the end portion 3 with the battery 22 has a maximum thickness of about 5.5 mm, the middle portion 7 has a maximum thickness of about 3.0 mm, and the end portion 3 with the charging coil 21 has a maximum thickness of about 3.3 mm. In a second example illustrating the thickness of the front portion 92 of the mouth guard 1, with the first layer 82 being 3 mm in thickness and the second layer 84 being 3 mm in thickness, the end portion 3 with the battery 22 has a maximum thickness of about 6.1 mm, the middle portion 7 has a maximum thickness of about 3.7 mm, and the end portion 3 with the charging coil 21 has a maximum thickness of about 4.0 mm.

The overall thickness of the mouth guard 1 should not necessarily impact the data measured, but can be compensated for by algorithms or software when programming the processor 28 for the predetermined threshold levels. For example, when additional thickness of the mouth guard 1 is used, it dampens the measured impact force. In other words, the mouth guard 1 is effectively reducing the amount of impact/force on the skull and brain by a small amount.

FIG. 7 demonstrates the finished mouth guard 1 produced by the manufacturing process of FIG. 6. The mouth guard 1 includes a main body having the arched-shaped peripheral side surface 90 on its front portion 92 that generally follows the contour of the user's dental arch in the maxilla region. The front portion 92, which is comprised of the layering of the first layer 82, the PCBA 2, and the second layer 84, includes the electronic components. While most of those electronic components are facing inwardly toward the teeth (and not seen in FIG. 7), but the LED 10 faces the buccal direction and can be seen through the second layer 84 of plastic. The charging coil 21 is also seen on the front side 4 of the PCBA 2. The center tab 11 that receives the high-G accelerometer 30 is positioned in a location that is substantially aligned with the midline of the upper incisors of the user. The mouth guard includes an arch-shaped depressed portion 94 connected to the front portion 92 that is sized and shaped to receive teeth of the user to help retain the mouth guard in a fixed position in the mouth. A palate portion 96 of the mouth guard 1 engages the roof of the mouth. Given the drive to produce a device that minimizes the size, permits accurate data collection, and provides user comfort, the mouth guard 1 of the present invention works better with the aforementioned forming process. Though it is possible to use a mouth guard created from a one-size-fits-all approach (such as boil-and-bite mouth guards) with the present invention, the aforementioned forming process is the preferred method of manufacturing.

The first layer 82 can be made of a clear or colored material. The second layer 84 is preferably clear in the region of the LED 10 so that it can be observed by others, but other regions of the second layer 84 can be colored. If no LED 10 is present in the mouth guard 1, then both layers 82 and 84 can be colored and opaque.

As shown in FIG. 8, a remote receiver in the smart device 50 may act with a local app (e.g., software application) to receive and display real-time data or information from the mouth guard(s) 1 via the communication system (e.g., Bluetooth transceiver 29). Preferably, the remote smart device 50 includes a display 52 that permits the displaying of certain types of impact information from a specific mouth guard 1 associated with a user (or multiple mouth guards from several users). For example, the display 52 may include a relative time indicator 54 that may demonstrate the timing of a recent impact event. Furthermore, the display 52 may include impact data 56, such as acceleration and force (linear and/or rotational). Further, a risk calculation 58 may be displayed. The risk calculation may be determined either locally or remotely, and is based predetermined impact thresholds unique to each user based on his or her biometric information. The risk calculation can also be dynamically generated in that it is based on series of impacts within a certain period of time. Accordingly, the present invention contemplates that when the mouth guard 1 senses a force exceeding a certain threshold, there are multiple options for notifications: (i) to the user via the feedback from the notification component 36, (ii) to other individuals near the user via the LED 10, and (iii) to more-remotely situated individuals via the display 52 of the smart device 50.

As noted above, the smart device 50 also permits the user to communicate with the mouth guard 1. For example, the user can use the smart device 50 to select his or her activity for the day such that processor 28 then selects the corresponding impact force threshold data for that particular activity. The user can use the smart device 50 to indicate the occurrence of a prior concussion, such that processor 28 then selects a reduced impact threshold data. Or, in another alternative, the user can dictate the reduced value of the impact threshold by indicating a specific percent reduction in the threshold if he or she desires to be notified of lesser hits to stay far away from injury. The smart device 50 may, in response to the user's command, download force data from memory 34 on the PCBA 2, and then send instruction to erase the prior force data or segments of the prior force data.

The smart device 50 is preferably in communication with a remote storage (e.g., the cloud) that contains the various types of threshold data for all users or a subset of users (e.g., football players). As better and more precise threshold data is learned and stored in the remote storage, the smart device 50 downloads that updated data, which can then be transmitted to the user's mouth guard 1 via the Bluetooth connection. In another example, if the user decides to try another activity, such as rugby, the smart device 50 can download the rugby threshold data from the remote storage and then transmit that rugby threshold data to the customized mouth guard 1 for that user.

The present invention further contemplates a false impact detection algorithm and methodology. False impacts can be detected in a number of different ways. In some embodiments, false impacts can be detected by using the mouth guard 1 to determine if the mouth guard 1 was in the user's mouth when an impact is detected. In these embodiments, the mouth guard 1 will generally include one or more sensors that generate presence data indicative of whether the mouth guard 1 is currently present in the user's mouth. If data generated by the sensors 30, 31, and/or 32 indicates that an impact occurred, but the presence data indicates that the mouth guard 1 was not in the user's mouth when that impact supposedly occurred, it can be determined that that impact was a false impact.

In some embodiments, the one or more sensors configured to detect whether the mouth guard 1 is in the user's mouth includes one or more optical sensors. Generally, the optical sensor(s) is configured to generate data representative of the ambient light in the area around the optical sensor. This data can be analyzed to determine the level or intensity of the light in the area around the optical sensor (e.g., the brightness of the area around the optical sensor). If the brightness of the area around the optical sensor(s) is greater than (and/or equal to) a threshold brightness, it can be determined that the mouth guard 1 is not in the user's mouth. If the brightness of the area around the optical sensor(s) is less than (and/or equal to) a threshold brightness, it can be determined that the mouth guard is in the user's mouth, and the user's mouth is blocking ambient light from reaching the optical sensor(s). In some cases, the optical sensor(s) is used to determine the average brightness of the ambient light over a time period. If the average brightness is greater than (and/or equal to) a threshold brightness, it can be determined that the mouth guard 1 is not in the user's mouth. If the average brightness is less than (and/or equal to) a threshold brightness, it can be determined that the mouth guard 1 is in the user's mouth. For example, if the user yawns while the mouth guard 1 is in the user's mouth, the brightness of the ambient light as measured by the optical sensor(s) may temporarily increase above the threshold, which could indicate that the mouth guard 1 is not in the user's mouth, and data generated by the sensors 30-32 during this period can be disregarded. By utilizing the average brightness over a time period, data generated by the sensors 30-32 will not be disregarded. The time period over which the average brightness is measured can be any suitable time period, such as 1 second, 2 seconds, 5 seconds, 10 seconds, 20 seconds, 30 seconds, etc.

In some embodiments, the one or more sensors configured to detect whether the mouth guard 1 is in the user's mouth includes one or more infrared (IR) sensors. The IR sensor(s) can be used to generate data indicative of the density of a material that the IR sensor(s) is in close proximity to (e.g., is adjacent to or next to). If the mouth guard 1 is in the user's mouth, the data generated by the IR sensor(s) may indicate that the IR sensor(s) near the user's teeth or gums, and it can be determined that the mouth guard 1 is in the user's mouth. Similar to the optical sensor(s), the IR sensor(s) could also be used to detect an average density over a time period, to prevent erroneous out-of-mouth detection.

In some embodiments, the one or more sensor configured to detect whether the mouth guard 1 is in the user's mouth includes one or more temperature sensors that generate data indicative of the temperature of the area around the temperature sensor. Generally, the ambient temperature in the area around the temperature sensor will be warmer when the mouth guard 1 is in the user's mouth. Thus, if the temperature detected by the temperature sensor(s) is greater than a temperature threshold, it can be determined that the mouth guard 1 is in the user's mouth. If the temperature detected by the temperature sensor(s) is less than a threshold temperature, it can be determined that the mouth guard is not in the user's mouth. The average temperature over a time period can also be used to determine whether the mouth guard 1 is in the user's mouth.

In some embodiments, multiple different types of sensors can be used to determine whether the mouth guard 1 is in the user's mouth. For example, the mouth guard 1 could include one or more optical sensors and one or more IR sensors; one or more optical sensors and one or more temperature sensors; one or more IR sensors and one or more temperature sensors; or one or more optical sensors, one or more IR sensors, and one or more temperature sensors. Additionally, other types of sensors could also be used (alone or in combination with optical/IR/temperature sensors) to determine whether the mouth guard 1 is in the user's mouth.

In some embodiments, false impacts can additionally or alternatively be detected by analyzing data generated by any combination of the sensors 30, 31, and 32 in the mouth guard 1. In some cases, the determination of whether an individual impact event is a true impact event or a false impact event is based solely on impact data generated by the sensors 30-32. For example, if data generated by the sensors 30-32 only weakly indicates that a true impact event occurred, the presence data can be used to aid in determining whether the impact was a true impact event or a false impact event. In other cases, the determination of whether an individual impact event is a true impact event or a false impact event is based on both impact data generated by the sensors 30-32, and the presence data generated by z,999

Preferably, the processor 28 receives and processes signals from the sensors 30, 31, 32 and use the algorithm, preferably stored as firmware (in the internal memory of the processor 28 or in the memory 34, to rule out false impact events while the mouth guard 1 is being used. The processor 28 identifies and labels any false impacts that have been recorded. False impacts are generally caused by various events, such as, placing and removing the mouth guard 1 to and from the user's mouth, irregular movement of the mouth guard 1 within the user's mouth, performing activities with the mouth guard 1 while not in the mouth, and chewing/teeth grinding on mouth guard 1.

As an initial part of the method for detecting false impacts, a variety of known false impact events are recorded by during uses of one or more mouth guards (e.g., mouth guard 1) by one or more users. Data from the false impact events may include testing from specific activities where the mouth-guard is commonly used, such as a team-sports session (e.g., high school football impacts recorded during games or practices) or individual competitive or recreational sessions (wrestling, boxing, grappling, biking, MMA fighting, skateboarding, etc.). Data can also be derived from laboratory sessions involving the mouth guard 1 (e.g., dropping the mouth guard 1 from heights, teeth grinding, gripping mouth guard with hand). It should be understood that a certain activity (e.g., football) is more likely to produce the same type of false impact events relative to other activities (e.g., biking or wrestling). Hence, the present invention contemplates unique algorithms for a certain activity, and players who are engaged in that activity may use the activity-specific algorithm for detecting false impact events with the mouth guard 1 while engaged in that specific activity. The recorded known false impact events can be used to generate the algorithms that will later be utilized by end-users (e.g., athletes, customers, etc.) to determine whether unknown impacts are true impacts or false impacts, and thus to calibrate the mouth guard

Each impact event is preferably validated against video footage taken during each of the sessions. Each recorded impact event is then categorized (e.g., true head impact, player was idle, mouth guard 1 not in mouth, etc.). Generally, data that is generated by the sensors 30, 31, and 32 will be time-stamped, to aid in correlating the generated data with the video footage. A final catalog of recorded impact events is preferably divided into three sets: (1) Training data, (2) Validation data, and (3) Held-out data. The data in these three sets of data will generally be labeled, such as “true impact,” “false impact,” etc. The training data set will be the largest containing about 60% of all labeled data, while the validation data set and the held-out data set will each contain about 20%. The proportion of validated impacts and false impacts is preferably the same across all types of sessions. These sets of data can be used to develop the standards for detecting future false impacts (e.g., determining whether an impact was a true impact or a false impact without the benefit of video review). In some embodiments, the data can be used to develop a newly chosen threshold or thresholds, and confirm that these thresholds will work to identify false impact events. In other embodiments, the data can be used to train a model to determine whether impact data represents a true impact or a false impact. Generally, the video validation is only used to generate the training data, validation data, and held-out data that is used to generate the algorithms utilized by the end-users, e.g., to develop and confirm new thresholds, and/or to train one or more models. False impacts detected during end-use of the mouth guard 1 (e.g., while an athlete is wearing the mouth guard 1 during an activity) do not need to be compared to video footage. However, in some cases, video comparison may be undertaken for certain purposes, as discussed in more detail herein.

To create a new threshold, the firmware has a fixed linear acceleration threshold. When the voltage associated with one of the sensors 30, 31, 32 crosses this threshold at any time, the firmware saves the previous 20 milliseconds (ms) and the next 80 ms of data as an impact. Using the training data set, preprocessed data is used on the raw data (sensor voltages) to extract features that the onboard sensors 30, 31, 32 have detected instead of the final processed data. For example, common features to be detected across the training data set include slope, duration of rising edge, and smoothness of initial rising slope. From the labeled false impact events from the training data set, the common features will be extracted based on the extracted feature we will develop a new threshold criterion or criteria.

For testing the new threshold criterion, the firmware undergoes simulation against triggering events, preferably using different possible threshold criterion. The validation data set of true and false impacts events is passed through this simulation, permitting true impact events to be detected and recorded while reducing the number of false impacts to be recorded with the mouth guard 1. The threshold or combination of thresholds that has the best reduction of false impact event recording without eliminating any true impact events from being recorded is chosen as the new standard threshold for use in the mouth guard 1. During use of the algorithm, features from the signals generated by the sensors in the mouth guard 1 can be analyzed. If the signals have features that are common for false impacts, the algorithm can determine that a given impact event was a false impact event. If the signals lack features that are common for false impacts, the algorithm can determine that a given impact event was a true impact event.

In a further embodiment, the false impact detection uses post-processed data that is input into a trained model. In this embodiment, proprietary filters determined from prior internal validation studies can be applied to the data generated by the sensors 30, 31, and 32. The proprietary filters may be general (e.g., they are applied to all impact data), or they could be activity-specific (e.g., a first set of filters is applied to impact data generated during a first type of sporting event, while a second set of filters is applied to impact data generated during a second type of sporting event). In this embodiment, a model is first trained using the training data set (e.g., impact data that is labeled, such as “true impact” or “false impact”). Once the model is trained, unlabeled data (e.g., data corresponding to impacts events where it is unknown whether that impact was a true impact or a false impact) can be input into the model, which determines whether the impact was a true impact or a false impact. Features from the impact data can be extracted and then input into the model, or the model can be trained to extract features from the impact data. In either case, the model is trained to determine whether the input data corresponds to a true impact or a false impact.

A variety of different models can be used. For example, in some embodiments, one or more of the following machine learning algorithms are used: random forests, support vector machine, k-means, neural networks, etc. Model training includes hyper-parameter optimization and/or other training techniques. After a model has been trained using the training data set, the model can then be evaluated using the validation data set, and given a single or several scores (e.g. ROC (also referred to as receiver operator characteristic), RMSE (also referred to as root mean square error), RMNS, F-Score). After a model is evaluated, changes can be made in an attempt to improve the model score by changing (add or removing) features or changing the model type itself (e.g. from a random forest to a neural network). This evaluation process is repeated until a final model (“Model Ensemble”) is chosen, which can be a combination of previous models. The final model is then evaluated, preferably using the final held-out data set mentioned above.

After the final model is acceptable, the algorithm can be implemented locally on the mouth guard 1 via the processor 28 and memory 24. Alternatively, this algorithm may be implemented as a background process to an App on the smart device 50 that is coupled to the mouth guard 1 or in a Cloud-based system. For example, the algorithm may be implemented in the Cloud-based system as a daily processing job that determines and/or removes false impact events from newly uploaded data across the platform associated with the user. In other words, the false impact detection occurs at a later time and removes the false impact events from the user's profile.

In some embodiments, different activities utilize activity-specific models trained using data from that activity. For example, to detect false impacts during a football game, a model that is trained using impact data generated during football games. This impact data can include data representing true impacts and false impacts. False impact data could be activity-nonspecific however (e.g., false impact data from dropping the mouth guard 1 could generally be used to train a model for any activity). Thus, after impact data is generated, a trained model that is specific to an activity can be selected from a plurality of available different models. In some cases, different activities will utilize different types of models to analyze impact data from those activities (e.g., a first activity uses a neural network and a second activity uses a support vector machine). In some cases, different activities will utilize identical types of model (e.g., both a first activity and a second activity will use the same type of model, such as a neural network).

In some embodiments, models can continually be trained using feedback data. For example, a trained model can analyze the impact data from a variety of different impact events, and determine whether each impact event represented a true impact or a false impact. At some time later, that same impact data can be labelled as representing a true impact or a false impact (or even some other outcome), and then used to train/update the model. Thus, the same data that the model initially operates on to identify a true impact or a false impact can also later be used to update the model, thus improving the accuracy of the model.

In order to re-use impact data to update the model, the impact data will need to be labeled sometime after the model initially analyzes the impact data. In some cases, video footage correlated to the impact data can be analyzed to determine whether the impact data represented a true impact or a false impact, and the impact data can then be labelled and used to update the model. In other cases, feedback data can be received that confirms whether the impact data represented a true impact or a false impact. This feedback data could be user feedback that is generated by the user of the mouth guard 1. For example, after a sporting event (e.g., a football game), a user (e.g., a participant in the sporting event) can review impact data generated by their mouth guard during the event, and confirm whether any of the impact events were true impacts or false impact. This feedback data can be used to label the impact data from the sporting event, which can in turn be used to update the model.

In some embodiments, the model can be updated using a Cloud-based system, and the algorithm with the updated model can be sent to the individual mouth guards. In other embodiment, the model is implemented only in the Cloud-based system, and the algorithm is not sent to the individual mouth guards after being updated.

Labeling the previously unlabeled impact data can generally be done at any time. In some embodiments, the model is used in real time during a sporting event, and is periodically updated using now-labeled impact data. The updating could happen on any suitable schedule, e.g., daily, weekly, monthly, after every sporting event, after every two sporting events, after every five sporting events, etc. The updating could also occur on a smaller time frame. For example, the model could be updated during a break in sporting event (e.g., between quarters or periods, at halftime, etc.). In some embodiments, the model is updated as soon as previously unlabeled impact data can be labeled.

After an impact event has been determined to be a true impact or a false impact, a variety of different actions can be taken. In some cases, the user and/or another person can be notified that an impact was a false impact or a true impact. The other person could be a coach, a parent, person assigned to monitor one or more mouth guards during a sporting event (e.g., a member of a team training staff), or some other person. This notification can allow the user and other the other person to adjust the user's participation in the sporting event based on the determined true or false impact. The determination of a true impact or a false impact can also be used to adjust a score or other evaluation of impacts that the user has received during the sporting, or during some other time period.

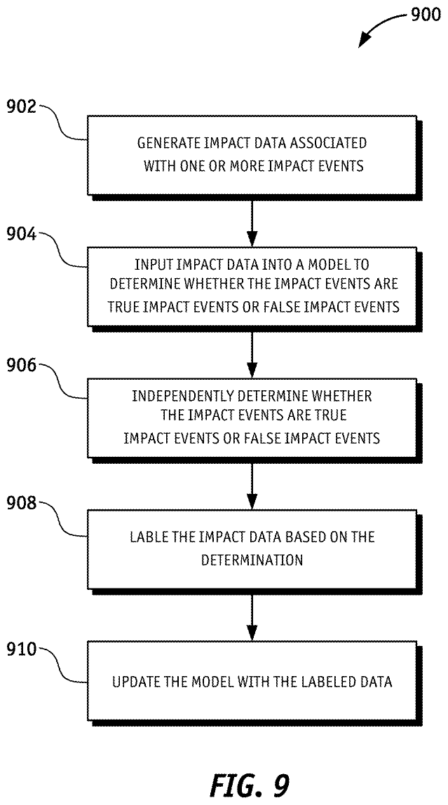

FIG. 9 illustrates a method 900 for detecting false impacts. Generally, method 900 can be implemented using any suitable processing device or combination of processing devices. For example, method 900 can be implemented using components of the mouth guard 1, such as the processor 28 and the memory 34. Method 900 could also be implemented using a system external to the mouth guard 1, such as a Cloud-based system. Method 900 could further be implemented using both the mouth guard 1 and an external system. For example, mouth guard 1 could perform a portion of the steps of method 900, while the external system could perform the remaining steps of method 900.

Step 902 of method 900 includes generating impact data that is associated with one or more impact events. In most cases, the impact data is generated using one or more mouth guards, and is representative of one or more impact events experienced by each mouth guard. These impact events could be true impact events or false impact events. Each of the mouth guards (one or more of which can be the same as or similar to mouth guard 1) will generally correspond to a respective user. Any data generated by an individual mouth guard (e.g., data generated by any combination of the sensors 30, 31, and 32) will thus be associated with a respective user.

Step 904 of method 900 includes inputting the impact data into a model to determine whether the impact events experienced by one or more of the mouth guards are true impact events or false impact events. Generally, a set of impact data will be generated by each mouth guard when that mouth guard experiences an impact event. Any amount of impact data generated by any number of mouth guards can be input into the model to determine whether any impact events experienced by the mouth guards were true impact events or false impact events. Because each mouth guard corresponds to a respective user, step 904 includes determining whether the user experienced a true impact or not. In some cases, the trained model is a trained machine learning algorithm, such as a neural network or a support vector machine. In some embodiments, method 900 can further include generating presence data that is indicative of whether one or more of the mouth guards is present within the mouth of the respective user when the impact data is generated. The determination of whether the impact event was a true impact event or a false impact event can then be based at least in part on this presence data.

In some embodiments, inputting the impact data into the model includes selecting the model from a plurality of models. The selection of the model can be based at least in part on the type of activity engaged in by the users. For example, impact data generated by users participating in a first type of activity (e.g., football) may utilize a first model that is trained with data generated from users participating in that first type of activity, while impact data generated by users participating in a second type of activity (e.g., soccer) may utilize a second model that is trained with data generated from users participating in that second type of activity. In some cases, different activities may utilize different types of models (e.g., a neural network versus a support vector machine) that are trained for their respective type of activity. In other cases, different activities may each utilize the same type of model, that has been trained for that type of activity.

In some embodiments, step 902 can additionally or alternatively include analyzing the impact data in a different manner to determine if the impact event was a true impact event or a false impact event. For example, the impact data can be compared to thresholds from prior recorded false impact events (sometimes referred to as non-impact events) to determine whether the impact event was a true impact event or a false impact event.

In some embodiments, method 900 further includes steps 906-910. Step 906 includes independently determining whether the impact events represented by the impact data were true impact events or false impact events. In some cases, feedback data can be received that is indicative of whether the impact event was a true impact event or a false impact event. Generally, the feedback data will be an indication as to whether each impact event was a true impact event or a false impact event The feedback data could be generated by the users themselves (e.g., the participants in the activity that wore the mouth guards) after the activity. The users could provide feedback data in a variety of manners. For example, the users could affirmatively provide information about the impacts they received during the activity. In another example, the users could provide information in response to being asked questions about impacts they received during the activity. The feedback data can additionally or alternatively be input by a third party, such as a coach or trainer who was present during the activity. Other persons could also provide the feedback data. In some cases, video of the activity (e.g., video of a football game or practice) can be reviewed (by the users and/or by other persons) to determine whether each impact event was a true impact event or a false impact event. The impact data will generally be timestamped so that the impact data can be correlated with the video.

Step 908 of method 900 includes labeling the impact data based on the independent determination undertaken in step 906. Generally, any portion of the impact data that corresponds to a single distinct impact event can be labeled as representing a true impact event or a false impact event. Labeling the impact data creates training data (e.g., data that is independently associates with an outcome or a category) that can be used to update the trained model. Step 910 of method 900 includes updating the model with the training data (e.g., the now-labeled impact data that was previously unlabeled), so that the model can better analyze unlabeled impact data in the future. The model can be updated on any suitable timeframe. Thus, the impact data that was analyzed to determine whether impact events experienced by the mouth guard were true impact events or false impact events, can later be used to improve the model's ability to determine whether an impact event was a true impact event or a false impact event.

These embodiments and obvious variations thereof is contemplated as falling within the spirit and scope of the claimed invention, which is set forth in the following claims. Moreover, the present concepts expressly include any and all combinations and subcombinations of the preceding elements and aspects.

1. A method for detecting false impacts, comprising:
generating impact data using one or more mouth guards, each of the one or more mouth guards corresponding to a respective user of one or more users; and
based at least in part on the impact data, determining whether an impact event experienced by at least one of the one or more mouth guards is a true impact event or a false impact event.

2. The method of claim 1, wherein the determining includes inputting at least a portion of the impact data into a model, the model being configured to analyze the impact data and determine whether the impact event is a true impact event or a false impact event.

3. The method of claim 2, wherein the model is a trained machine learning algorithm.

4. The method of claim 3, wherein trained machine learning algorithm is a support vector machine or a neural network.

5. The method of claim 1, further comprising:
selecting a model from a plurality of models; and
inputting at least a portion of the impact data into the selected model, the model being configured to analyze the impact data and determine whether the impact event is a true impact event or a false impact event.

6. The method of claim 5, wherein the selection of the model is based at least in part on a type of activity engaged in by the at least one of the one or users.

7. The method of claim 6, wherein the selected model is previously trained using training data generated during one or more prior instances of the type of activity engaged in by the at least one of the one or more users.

8. The method of claim 1, further comprising:
selecting a first model from a plurality of models;
inputting first impact data generated from a first mouth guard into the first model, the first impact data representing one or more impact events experienced by a first user engaged in a first activity;
selecting a second model from a plurality of models; and
inputting second impact data generated from a second mouth guard into the second model, the second impact data representing one or more impact events experienced by a second user engaged in a second activity,
wherein the first activity is different than the second activity, and the first model is different than the second model.

9. The method of claim 8, wherein the first model is a first type of machine learning algorithm, and the second model is a second type of machine learning model.

10. The method of claim 8, wherein the first model and the second model are identical types of machine learning algorithms.

11. The method of claim 8, wherein the first model is previously trained using first training data generated during one more prior instances of the first activity, and the second model is previously trained using second training data generated during one or more prior instances of the second activity.

12. The method of claim 1, further comprising generating presence data indicative of whether each of the one or more mouth guards is present within a mouth of the respective one or more users when the impact data is generated, the determining being based at least in part on the presence data.

13. A method for detecting false impacts, comprising:
generating impact data using one or more mouth guards, each of the one or more mouth guards corresponding to a respective user of one or more users;
inputting at least a portion of the impact data into a model, the model being configured to analyze the impact data and determine whether an impact event experienced by at least one of the one or more mouth guards is a true impact event or a false impact event;
independently determining whether the impact event is a true impact event or a false impact event;
labeling the portion of the impact data as representing a true impact event or a false impact event to thereby form training data; and
updating the model using the training data.

14. The method of claim 13, further comprising:
receiving feedback data associated with the impact event; and
using the feedback data to determine whether the impact event is a true impact event or a false impact event.

15. The method of claim 14, wherein the feedback data is generated by the one or more users.

16. The method of claim 14, wherein the feedback data is generated after a video review of the impact event.

17. A mouth guard system for detection impact forces, comprising:
a main body comprised of flexible material and having a front portion with a generally arched-shaped peripheral side for facing the buccal region of a mouth of a user, the main body further including a depressed portion adjacent to the front portion that is sized and shaped to receive teeth of the user;
at least one sensor for detecting a force and being embedded in the flexible material, the at least one sensor being located within the front portion; and
a memory device for storing a false impact detection algorithm to determine false impacts to the user, the false impact detection algorithm determining an impact threshold based on prior recorded non-impact events from one or more users.

18. The method of claim 1, wherein the determination of whether the impact event experienced by the at least one of the one or more mouth guards is a true impact event or a false impact event is unique to the activity in which a user of the at least one of the one or more mouth guards is engaged.

19. The method of claim 1, further comprising:
detecting at least a first impact event experienced by one of the one or more mouth guards with at least one of a linear force sensor and a rotational force sensor embedded in the one of the one or more mouth guards, the detection being based at least in part on the generated impact data;
determining whether a force associated with the first impact event exceeds a predetermined threshold;
determining whether signals from the sensors associated with the force has features that are common for false impacts;
in response to the signals having the features common for false impacts, determining the first impact event is a false impact event; and
in response to the signals lacking the features common for false impacts, determining the first impact event is a true impact event.

20. The method of claim 19, wherein the common features include slope of the signals, duration of rising edge of the signals, and smoothness of initial rising slope of the signals.
